(19) United States
(12) Patent Application Publication
(10) Pub. No.: US 2017/0333112 A1
(43) Pub. Date: Nov. 23, 2017

(54) Optimizing Total Energy Delivered in Nanosecond Pulses for Triggering Apoptosis in Cultured Cells

(71) Applicant: Pulse Biosciences, Inc., Burlingame, CA (US)

(72) Inventors: Richard L. Nuccitelli, Millbrae, CA (US); Zachary R. Mallon, Richmond, CA (US); Amanda H. McDaniel, San Bruno, CA (US); David J. Danitz, San Jose, CA (US); Brian G. Athos, San Francisco, CA (US); Mark P. Kreis, San Francisco, CA (US); Darrin R. Uecker, San Mateo, CA (US); Pamela S. Nuccitelli, Millbrae, CA (US)

(73) Assignee: Pulse Biosciences, Inc., Burlingame, CA (US)

(21) Appl. No.: 15/599,679

(22) Filed: May 19, 2017

Related U.S. Application Data
(60) Provisional application No. 62/339,566, filed on May 20, 2016.

(51) Int. Cl. A61B 18/14; A61B 5/055; A61N 1/32; A61B 18/00

(52) U.S. Cl. CPC A61B 18/14; A61N 1/327; A61B 5/055; A61B 2018/00613; A61B 2018/1405

(57) ABSTRACT

An optimization of electrical characteristics for treatments of tumor or other abnormal cells in culture with sub-microsecond, high-electric field electrical pulses is disclosed. The voltages, pulse widths, and number of pulses are chosen such that the treatment energy is 10-20 J/mL. That is, U=n*Δt*V*I/volume is 10-20 J/mL, in which n is the number of pulses, Δt is the duration of each pulse, V is the voltage, I is current, and volume is the area of parallel electrodes times the distance between them. V divided by the distance between the electrodes can be in an effective range of 6 kV/cm to 30 kV/cm, 60 kV/cm, 100 kV/cm, or higher intensities. Rows of needle electrodes, blade electrodes, or other configurations of electrodes can approximate parallel electrodes.

### CROSS-REFERENCES TO RELATED APPLICATIONS

This application claims the benefit of U.S. Provisional Application No. 62/339,566, filed May 20, 2016, which is hereby incorporated by reference in its entirety for all purposes.

### STATEMENT AS TO RIGHTS TO INVENTIONS MADE UNDER FEDERALLY SPONSORED RESEARCH AND DEVELOPMENT

Not Applicable

### BACKGROUND

### 1. Field of the Invention

The present application generally relates to electroporation of cells, specifically those for initiating apoptosis in cells by means of sub-microsecond, high electric field electrical pulses at particular total energies.

### 2. Description of the Related Art

An “abnormal cell” can be defined as any tumor cell or cell from unwanted growth of tissue from a subject, or as otherwise known in the art. A “subject” may include a human, mammal (that is non-human), or other affected animal.

A “tumor” can be defined as any neoplasm or abnormal, unwanted growth of tissue on or within a subject, or as otherwise known in the art. A tumor can include a collection of one or more cells exhibiting abnormal growth. There are many types of tumors. A malignant tumor is cancerous, a pre-malignant tumor is precancerous, and a benign tumor is noncancerous. Examples of tumors include a benign prostatic hyperplasia (BPH), uterine fibroid, pancreatic carcinoma, liver carcinoma, kidney carcinoma, colon carcinoma, pre-basal cell carcinoma, and tissue associated with Barrett's esophagus.

Periodic sub-microsecond electrical pulses with high electric fields (e.g., greater than 5 kilovolts per centimeter (kV/cm)) have been shown to kill tumors in laboratory mice. The tumors die because the nanosecond pulsed electric fields apparently initiate apoptosis in their tumor cells, a programmed cell death.

The death of the tumor cells occurs without substantially affecting normal cells in surrounding tissue. Although they are at very high voltages, the electrical pulses do not inject enough energy to cause a rise in temperature of more than a few degrees because they are of extremely short durations. Nearby cells do not need to conduct much thermal energy away from the treatment zone because not much thermal energy is created. Unlike with thermally ablative medical instruments, the tissue simply does not heat up very much.

Unlike systemic drugs, electrical treatments stay local to the region being treated. The area treated is typically within a small volume between two or more electrodes. Therefore, side effects in other areas of the body are virtually nonexistent. Any stray electrical currents are small, and they spread out in the surrounding tissue.

A “nanosecond pulsed electric field,” sometimes abbreviated as nsPEF, includes an electric field with a pulse width of between 0.1 nanoseconds (ns) to 1000 nanoseconds, or as otherwise known in the art. It is sometimes referred to as sub-microsecond pulsed electric field. NsPEFs often have high peak voltages, such as 10 kilovolts per centimeter (kV/cm), 20 kV/cm, to 500 kV/cm. Treatment of biological cells with nsPEF often uses a multitude of periodic pulses at a frequency ranging from 0.1 pulses per second (pps or Hz) to 10,000 pps.

An example of nsPEF applied to biological cells is shown and described in U.S. Pat. No. 6,326,177 (to Schoenbach et al.), which is incorporated herein by reference in its entirety for all purposes.

Studies have subjected tumors to nanosecond pulsed electric fields of various pulse widths, frequencies, duty cycles, electric fields, numbers of pulses, and daily and weekly treatment regimes. Different types of tumors, sizes of tumors, cell lines, and other samples have been treated. All these parameters seem to affect results. Sometimes the treatments result in apoptosis, which is desired. Other times, the treatments result in necrosis. Other times, there is no effect. It is quite unpredictable. More voltage does not necessarily result in more apoptosis. More pulses does not necessarily result in more apoptosis. In fact, more voltage and more pulses can have the opposite effect. There are so many variables, and results are so time consuming to tedious to collect, that determining effective electrical parameters is hit-or-miss. This is especially troublesome in transitioning from animal models to humans.

While more research time and laboratory animal studies may conceivably lead to optimization of safe, effective electrical parameters for treating certain animals' tumors, the same studies cannot be performed with human subjects. This has held back the very promising field of nanosecond pulsed electric field treatments from making the jump from laboratory to human clinical trials.

There exists a need in the art for the safe and effective treatment of tumors and other abnormal growths in mammals and human subjects.

### BRIEF SUMMARY

Generally, treatments of tumor cells or other abnormal cells in culture with sub-microsecond, high-electric field electrical pulses is disclosed. The voltages, pulse widths, and number of pulses are chosen such that the actual electrical field delivered to the cells is between 6 kV/cm to 30 kV/cm, 60 kV/cm, 100 kV/cm, or higher intensities, and pulse width is below one microsecond, and the total treatment energy, modulated by the number of pulses, pulse widths, current limiter, or otherwise, is between 10-20 joules per milliliter (J/mL). For example, the number of pulses can be selected for a given voltage and pulse width so as to result in a total treatment energy of 15 J/mL.

Parallel plate electrodes, spaced apart like a capacitor, can produce a very uniform, linear electric field between them. Parallel plates are practical when cells can be suspended in a liquid and placed in a fluid holding region of a vessel between the plates. They also are practical when a tumor is near the surface of loose skin that can be pulled between the plates. Other electrode configurations may be more practical for in vivo treatments.

Needle electrodes produce less uniform electric fields. Instead of being uniform, the electric field tapers off with distance from a line between the needles and the needles themselves. Two needles produce an electric field with contour lines that bow out like a dog bone around the needles. Three or more needles produce more complicated fields. The polarities of the needles also affect the field lines.

Two rows of several needle electrodes approximates flat plates such that field lines between the rows are relatively straight if each row has an opposite polarity. The less distance between each needle in a row, and the more needles there, the more uniform the electric fields. Rows of needles may be used for cells in culture where there is a need for flow between needles in a row.

Some embodiments are related to a method of treating tumor cells or other abnormal cells. The method includes providing a volume between a pair of parallel plate electrodes, the parallel plate electrodes spaced at a distance from one another, selecting an electrical voltage V for the electrodes such that the electrical voltage V creates an electrical field in the volume, a magnitude of the electrical field between 6 kV/cm and 30 kV/cm, 60 kV/cm, 100 kV/cm, or higher intensities, placing tumor cells, that may be in a liquid, into a vessel having a fluid holding region or otherwise within the volume, determining an electrical current I produced by the electrical voltage through the tumor cells in the liquid, ascertaining a number of pulses n of the electrical voltage V such that an electrical energy density U is between 10 J/mL and 20 J/mL, wherein Δt is a duration of each pulse of the pulses, wherein n=U/(Δt*V*I/volume), and applying the number of pulses to the electrodes, thereby treating the volume and initiating apoptosis in the tumor cells.

The method can include changing the number of pulses n, during the applying, based on a feedback measurement of at least one prior pulse. The changing can results in the number of pulses n becoming fewer or larger. The duration of each pulse Δt can be changed during the applying based on a feedback measurement of at least one prior pulse. The electrical voltage V can be changed during the applying based on a feedback measurement of at least one prior pulse.

The method can include energizing the electrodes with the electrical voltage and measuring the electrical current I. The method can include looking up the electrical current I from a memory.

The parallel plate electrodes can each have a surface area of ‘a’ and, a distance between them being d, the volume being calculated by a*d. The vessel can have a fluid holding region within the volume between the pair of electrodes is an electroporation cuvette. The electroporation cuvette can incorporate the parallel plate electrodes, each electrode having a surface area ‘a’ of 2.1 cm², the cuvette having a distance d of 0.4 cm between the electrodes for a volume of 0.84 mL. Each pulse can have a duration Δt of 100 ns, and the pulses are applied at 0.1 pulses per second (pps) to 10 pps. The tumor cells can be in a liquid culture.

The method can include drawing the abnormal cells from a human or animal subject during a biopsy, dispersing the abnormal cells in the liquid, and reintroducing the abnormal cells, after the initiation of apoptosis, into the subject.

The method can include establishing a specific magnitude of the electrical field between 6 kV/cm and 100 kV/cm based on a type of the abnormal cells.

Some embodiments are related to a method of treating tumor cells or other abnormal cells, including providing at least two rows of needle electrodes, blade electrodes, or other configurations of electrodes, the rows of electrodes spaced at a distance from one another, selecting an electrical voltage V for the electrodes such that the electrical voltage V creates an electrical field in a volume between the rows of electrodes, a magnitude of the electrical field between 6 kV/cm and 30 kV/cm, 60 kV/cm, 100 kV/cm, or higher intensities, placing tumor cells that are in a liquid into a fluid within the volume, determining an electrical current I produced by the electrical voltage through the tumor cells in the liquid, ascertaining a number of pulses n of the electrical voltage V such that an electrical energy density U is between 10 J/mL and 20 J/mL, wherein Δt is a duration of each pulse of the pulses, wherein U=n*Δt*V*I/volume, and applying the number of pulses to the electrodes, thereby treating the volume and initiating apoptosis in the tumor cells.

The method can include changing the number of pulses n, during the applying, based on a feedback measurement of at least one prior pulse. The changing can results in the number of pulses n becoming fewer or larger. The duration of each pulse Δt can be changed during the applying based on a feedback measurement of at least one prior pulse. The electrical voltage V can be changed during the applying based on a feedback measurement of at least one prior pulse.

The method can include energizing the electrodes with the electrical voltage and measuring the electrical current I. The method can include looking up the electrical current I from a memory.

The method can include drawing the abnormal cells from a subject during a biopsy, dispersing the abnormal cells in the liquid, and reintroducing the abnormal cells, after the initiation of apoptosis, into the subject.

The method can include establishing a specific magnitude of the electrical field between 6 kV/cm and 100 kV/cm based on a type of the abnormal cells.

### BRIEF DESCRIPTION OF THE DRAWINGS

FIG. 1 illustrates cells in culture being treated in an electroporation cuvette in accordance with an embodiment.

FIG. 2 illustrates typical voltage and current oscilloscope traces of a 10 kV/cm pulse into a cuvette with a 4 mm gap and 20 ohm impedance in accordance with an embodiment.

FIG. 3 illustrates typical voltage and current oscilloscope traces of a 20 kV/cm pulse into cuvette with a 4 mm gap and 20 ohm impedance in accordance with an embodiment.

FIG. 4 is a chart of experimentally measured percent viable cells and current density versus measured conductivity in accordance with an embodiment.

FIG. 5 is a chart of experimentally measured percent viable cells versus total treatment energy in accordance with an embodiment.

FIG. 6 is a chart showing normalized levels of caspase 3 activation in different cell lines in accordance with an embodiment.

FIG. 7 is a chart showing normalized levels of caspase activation plotted by total treatment energy density in accordance with an embodiment.

FIG. 8 is a chart showing caspase activation in murine fibrosarcoma cells (MCA205) versus energy density applied using 100 ns pulses at the indicated field strengths in accordance with an embodiment.

FIG. 9 is a chart showing caspase activation in rat heptocellular carcinoma cells (McA-RH7777) versus energy applied using 100 ns pulses at the indicated field strengths in accordance with an embodiment.

FIG. 10 includes charts showing ATP released from MCA205, McA-RH7777, and Jurkat E6-1 cells at different numbers of pulses in accordance with an embodiment.

FIG. 11A is an isometric view of rows of needle electrodes in accordance with an embodiment.

FIG. 11B is a side view of an energized volume between the rows of needle electrodes of FIG. 11A.

FIG. 12 is a flowchart illustrating a process in accordance with an embodiment.

FIG. 13 is a flowchart illustrating a process in accordance with an embodiment.

### DETAILED DESCRIPTION

A non-thermal, drug-free tumor therapy referred to as Nano-Pulse Stimulation (NPS) can deliver ultrashort electric pulses to tumor cells to eliminate the tumor and inhibit secondary tumor growth. It has been hypothesized that the mechanism for inhibiting secondary tumor growth involves stimulating an adaptive immune response via an immunogenic form of apoptosis, commonly known as immunogenic cell death (ICD). ICD is characterized by the emission of danger-associated molecular patterns (DAMPs) that serve to recruit immune cells to the site of the tumor.

NPS pulses are fast enough (e.g., less than a millisecond) to penetrate almost all cells and organelles in the tumor before internal ions can rearrange to charge the membrane capacitance and screen out the cytoplasmic electric field. They are large enough to exert a sufficient force on dipole water molecules to drive them into lipid bilayers to generate transient nanopores in the plasma and organelle membranes. These nanopores allow calcium ions to flow down their concentration gradient and initiate immunogenic apoptosis when a sufficient number of pulses are applied.

NPS stimulates both caspase-3/7 activation indicative of apoptosis as well as the emission of three critical DAMPs: ecto-calreticulin (CRT), ATP, and HMGB1.

The initiation of apoptosis in cultured cells may be greatest at 15 kV/cm and require 50 A/cm². Reducing this current may inhibit cell death. This increase in cell apoptosis is non-linear with respect to energy per volume of cells and peaks at approximately 15-20 J/mL for field strengths of interest. 10 kV/cm or less and 30 kV/cm or more electric fields exhibited lower responses while 12 kV/cm and 15 kV/cm fields stimulate large amounts of caspase activation.

In studies conducted by the inventors, three DAMPs, twenty-four hours after treatment, have been measured. The expression of cell surface calreticulin (CRT) increased in an energy-dependent manner in the NPS treated samples. As a comparison, expression levels in NPS-treated samples reached or exceeded the expression levels in the majority of anthracycline-treated samples at energies between 25-50 J/mL. Anthracycline treatments are known to stimulate endoplasmic reticulum (ER) stress through indirect means. Similar to the caspase response at 3 hours, secreted adenosine triphosphate (ATP) peaked at 15 J/mL and then rapidly declined at 25 J/mL. High mobility group box 1 (HMGB1) protein release increased as treatment energy increased and reached levels comparable to the anthracycline-treated groups between 10-25 J/mL.

Many of the experiments described here were conducted with a fixed 100 ns pulse width generated by a pulse-forming network triggered by a spark gap. Some were conducted with a variable pulse width generator.

Caspase (cysteine-aspartic protease) 3 and caspase 7, referred to herein as caspase 3/7, are enzymes that cleave target proteins at specific aspartic acid amino acid locations. Their expression on cell surfaces often indicates apoptosis.

The steps leading to immunogenic apoptosis—and the energy required to trigger the activation of caspase 3 as a key protease in the apoptotic pathway—have been characterized.

Shown herein are measured amount of caspase 3/7 activation 3½ hours after NPES exposure in three malignant cell lines.

Cell Lines

For some studies described herein, an McA-RH7777 cell line was obtained from ATCC (formerly American Type Culture Collection) and was cultured in DMEM (Dulbecco's Modified Eagle Medium) containing 4 mM (millimolar) L-glutamine, 4500 mg/L glucose, 1 mM sodium pyruvate, and 1500 mg/L sodium bicarbonate, with 10% FBS (fetal bovine serum), and 1% penicillin streptomycin.

An MCA205 cell line was obtained from Andrew Weinberg, Providence Portland Medical Center, Portland, Oreg., U.S.A. and cultured in the same DMEM medium.

A Jurkat E6-1 cell line was obtained from ATCC and cultured in (Roswell Park Memorial Institute) RPMI 1640 (containing 2 mM L-glutamine, 10 mM HEPES (4-(2-hydroxyethyl)-1-piperazineethanesulfonic acid), 1 mM sodium pyruvate, 4500 mg/L glucose, and 1500 mg/L sodium bicarbonate), 10% FBS, and 1% Pen/Strep.

NPES Treatments

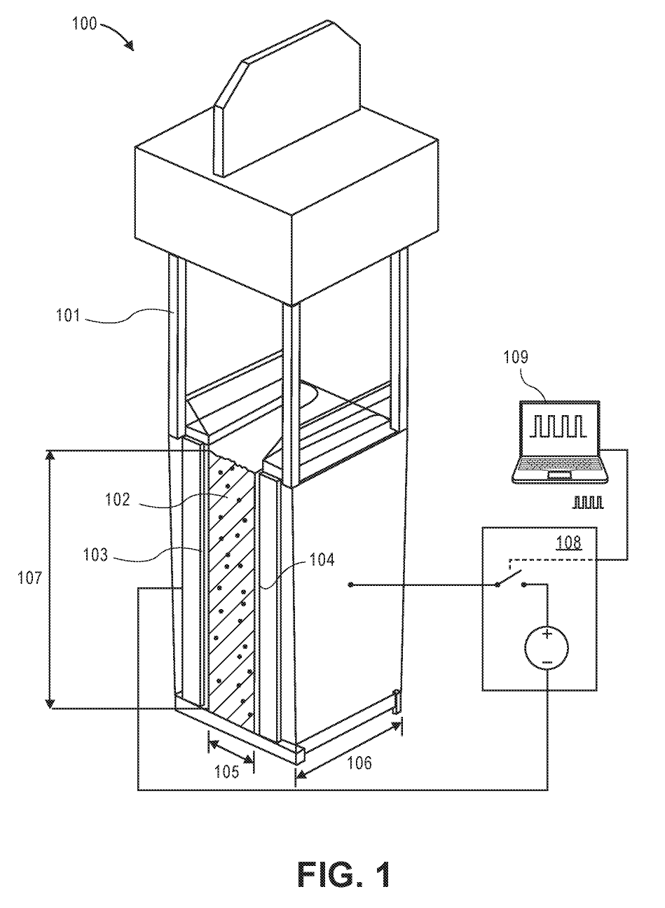

FIG. 1 illustrates cells in culture being treated in an electroporation cuvette. In system 100, electroporation cuvette 101 includes first planar electrode 103 and second planar electrode 104 configured as a pair of parallel plate electrodes. The parallel plate electrodes 103/104 are spaced at distance 105 from one another.

Cuvette 101 includes fluid holding region 102 in which abnormal cells in a liquid, e.g. cells in culture, are placed. The fluid fills the volume between parallel plates 103 and 104. Each planar electrode has area ‘a’. Area ‘a’ is height 107 multiplied by depth 106. Thus, the volume between the parallel plate electrodes is height 107×depth 106×distance 105.

The figure shows idealized circuit 108 for delivering high-voltage pulses. Idealized circuit 108, with a switch and a voltage source, provides a rapidly switched voltage to electrodes 103 and 104. The voltage, pulse width, and number of pulses can be controlled by computer 109. In some embodiments, the voltage may be adjusted during a train of pulses, i.e., before the train of pulses has ended. Similarly, pulse widths Δt may be adjusted, and so may the ultimate number of pulses in the train of pulses. These may be changed based on measured pulse widths, voltages, currents, or other feedback from measured electrical parameters of previous pulses.

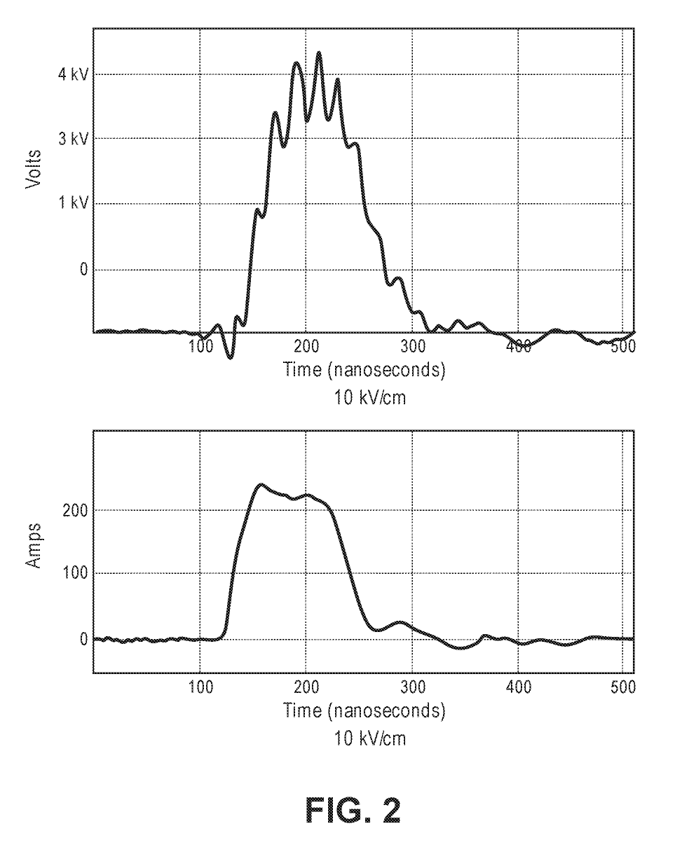

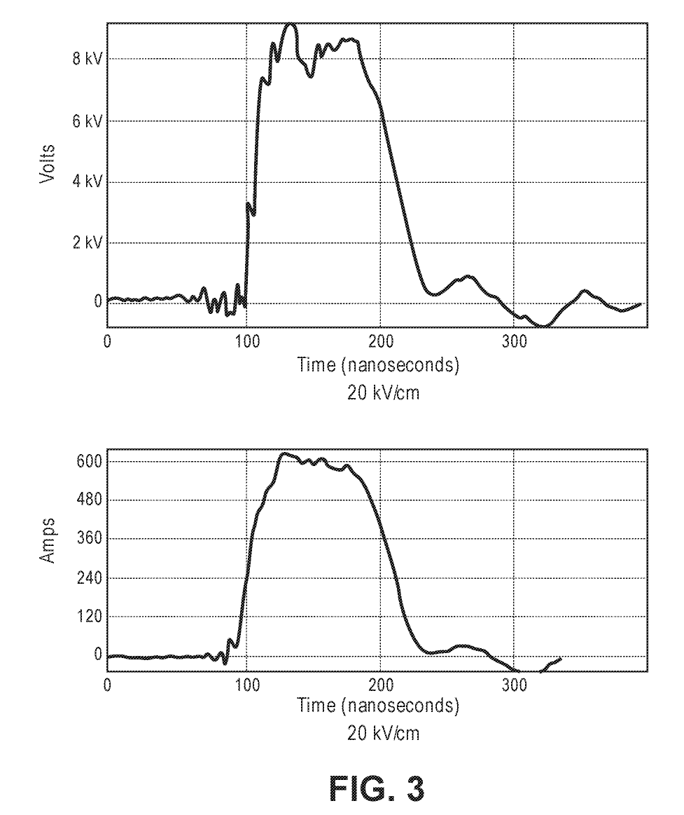

FIGS. 2-3 illustrate typical voltage and current oscilloscope traces of a 10 kV/cm pulse (FIG. 2) and 20 kV/cm pulse (FIG. 3) into a cuvette with a 4 mm gap and 20 ohm (Ω) impedance when filled with cells in solution.

As shown, actual pulses of these magnitudes and sub-microsecond durations may be hardly ideal, exhibiting overshoots, oscillations, and finite response times. In these conditions, engineering approximations may be made to an idea pulse. The “width” of each pulse may be considered the time that the voltage and/or current of the pulse is above a certain threshold. For example, the pulse width of the 8 kV pulse in FIG. 3 may be considered to be the time that the voltage is above 2 kV (25% of max), which is between 100 ns and 225 ns according to the figure. Non-ideal square pulses, and of course ideal pulses, are acceptable pulses for treatment.

NPS and Cell Viability

For many of the experimentally derived data herein, approximately one million cells were treated in 4 mm-wide electroporation cuvettes (Cell Projects# EP-104, Kent, United Kingdom) for treatments ranging from 6 kV/cm to 25 kV/cm. Three hundred and fifty thousand cells treated in 2 mm electroporation cuvettes (Life Technologies# P45050) were used for treatments of 30 kV/cm.

At a given treatment voltage, cells were treated with 100 ns pulses to meet the total treatment energy requirements ranging from 1.0 joule/mL to 80.0 joules/mL. The energy per mL is calculated as the product of the E field multiplied by the current applied, pulse duration and number of pulses divided by the area A of one side of a parallel plate electrode. That is, U=E*I*Δt*n/A.

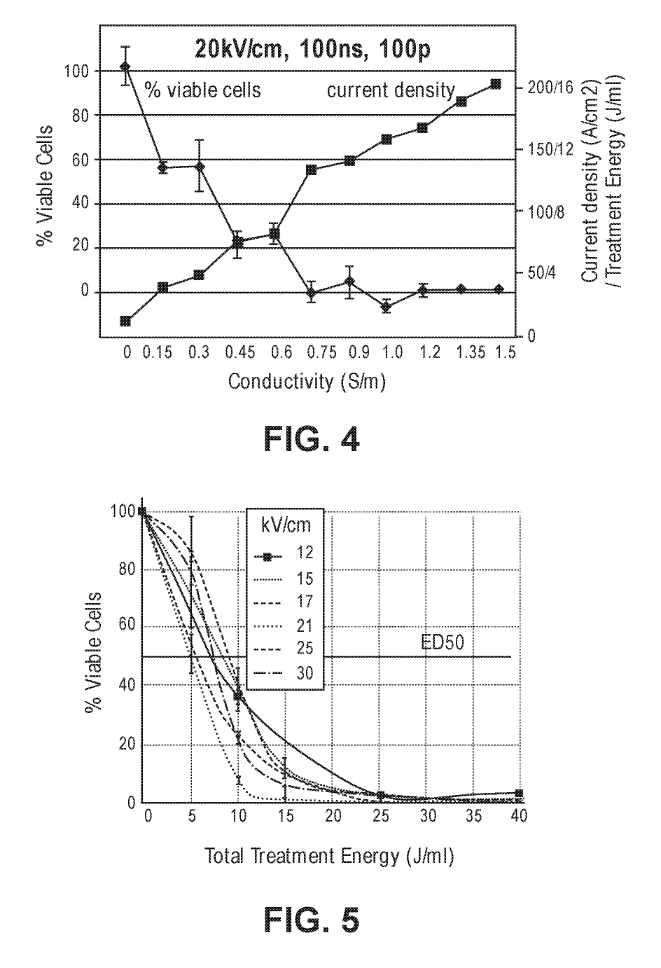

FIG. 4 is a chart of experimentally measured viable cells and current density versus measured conductivity. For these measurements, the McA-RH7777 rat hepatocellular cell line was used to study the dependence of cell viability on the total treatment energy in vitro using the PrestoBlue® assay, 3.5 hours after NPS treatment. Treatment energy was calculated by multiplying the number of pulses by the product of the applied voltage and current and pulse width.

By varying the conductivity of the medium, it has been confirmed that no significant cell death is observed from nanosecond pulsed electric field application when the current density flowing across the cells is below a threshold level. There is very little cell death in response to 100 pulses, 100 ns long, and 10-20 kV/cm when only 0 to 20 A/cm² is flowing across the cells.

Between 20 and 50 A/cm², the cell death begins to increase in a linear manner. Above 50 A/cm², the ablation efficiency increases sharply. It is nearly 100% for current densities greater than 100 A/cm², which corresponds to 8 J/mL (Energy (J/mL)=kV/cm*A/cm²*pulse duration*pulse number).

This imposes additional conditions on successful nanoelectroablation. To decrease the viability of cells, the field strength should exceed 10 kV/cm and current density should exceed 50 A/cm². This acute observation led the inventors to consider the energy delivered to the cell because it depends on both current and field strength.

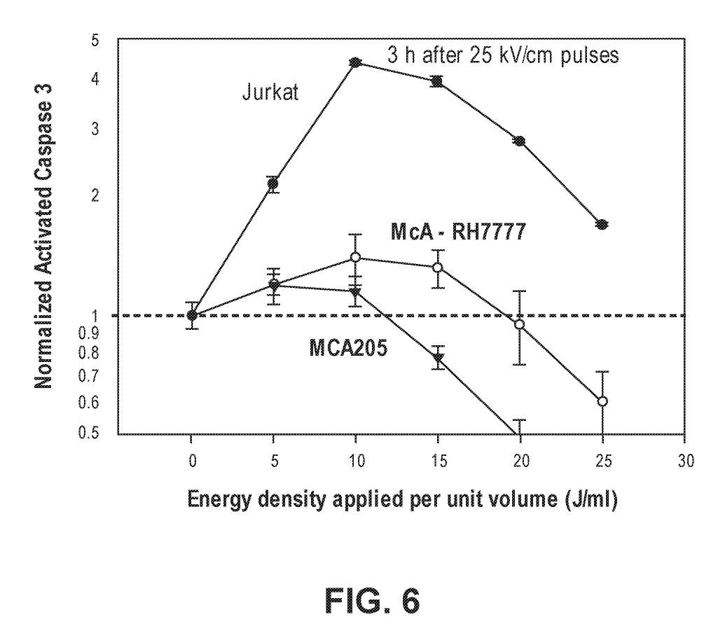

FIG. 5 is a chart of experimentally measured percent viable cells versus total treatment energy in accordance with an embodiment. Percent viable cells versus treatment energy are plotted for six different field strengths. As shown in the figure, when energy is held constant, the curves practically overlap. This demonstrates that field strength is not necessarily predictive of the ablative efficacy of NPS. In contrast, varying the total energy delivered during NPS is relatively highly predictive of ablative efficacy. The median effective dose for 50% of the population, ED50, for ablation is around 10-15 J/mL or 15-20 J/mL for a wide range of field strengths, including 12 kV/cm to 30 kV/cm. This may be applicable up to field strengths of 60 kV/cm. For example, electric field strengths of 6 kV/cm to 60 kV/cm, 100 kV/cm, or higher are envisioned.

Assessment of Caspase Activation

Activation of combined Caspase-3 and Caspase-7 was assessed using the Caspase-Glo® 3/7 Assay (Promega Corp. of Fitchburg, Wis., U.S.A.). Following NPES treatments, approximately fifteen thousand cells were plated onto three wells each of a 96 well assay plate containing pre-equilibrated media. Cells were then incubated for 3 hrs at 37° C., and 5% CO₂. Caspase-Glo reagent was then added to each well at a volume of 1:1 with cell media. Samples were then allowed to incubate for an additional 30 minutes at room temperature, protected from light, and with gentle agitation. Sample luminescence was then measured using the Molecular Devices (of Sunnyvale, Calif., U.S.A.) Spectramax i3 plate reader. Caspase activation caused by NPES treatment was normalized to unpulsed samples by dividing the raw luminescence units (RLUs) of pulsed samples by the RLU value of unpulsed controls.

Results

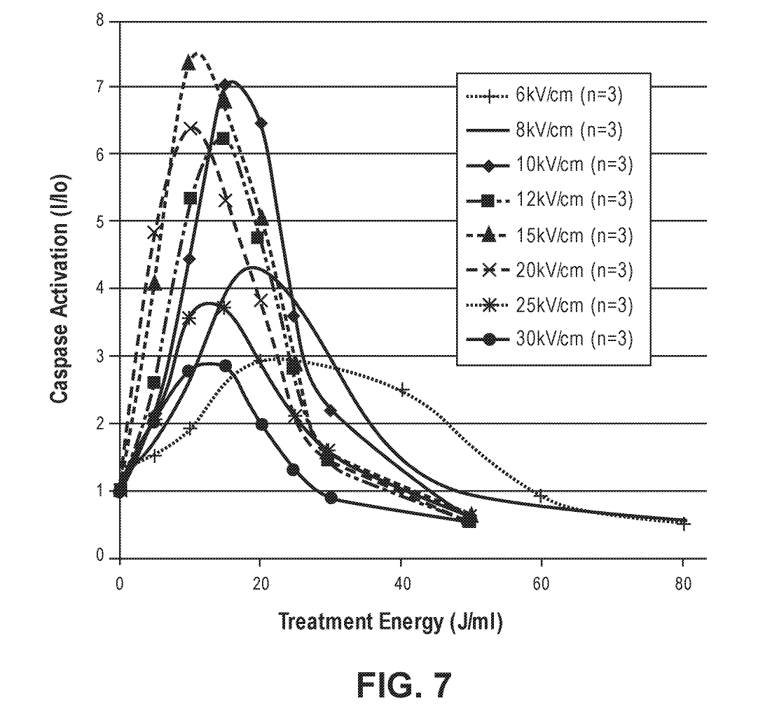

FIG. 6 is a chart showing normalized levels of caspase 3 activation in different cell lines in accordance with an embodiment. Three malignant cell lines are shown: 1) human Jurkat E6-1 T-lymphocytes; 2) murine MCA205 fibrosarcoma cells; and 3) rat McA-RH7777 hepatocarcinoma cells.

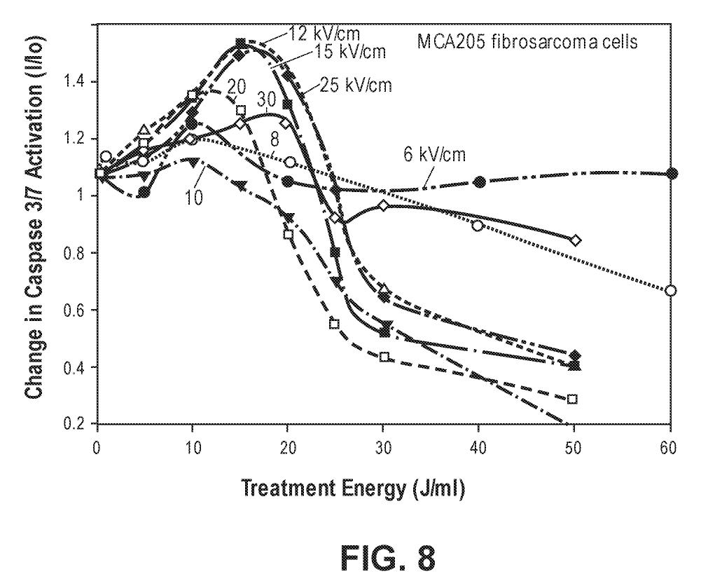

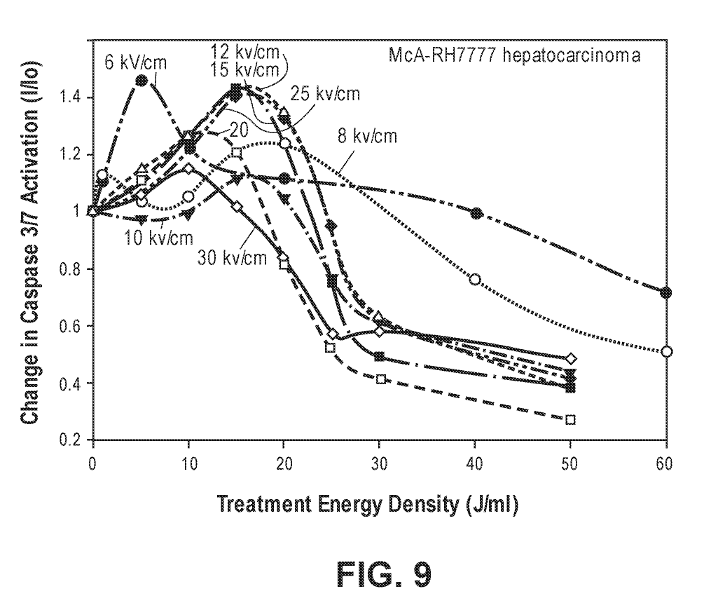

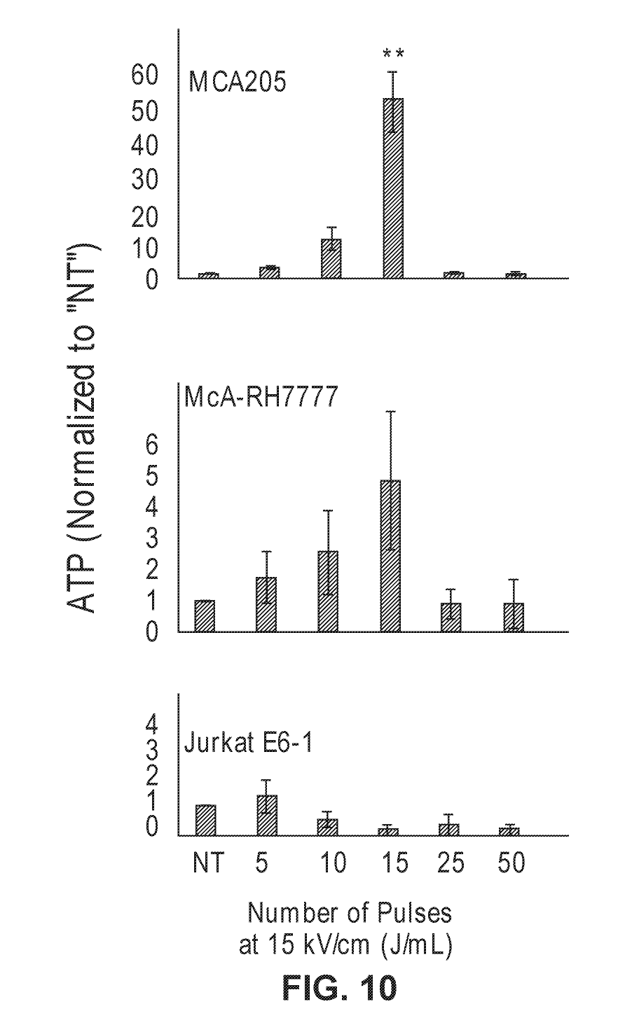

FIGS. 7-9 are charts showing normalized levels of caspase activation plotted by total treatment energy density. FIG. 7 shows caspase activation in human Jurkat cells. FIG. 8 shows caspase activation in murine fibrosarcoma cells (MCA205), and FIG. 9 shows caspase activation in rat heptocellular carcinoma cells (McA-RH7777).

The strongest response was observed in the human Jurkat cells (FIG. 7) where the levels of activated caspase increased to nearly 8-fold. The activation of caspase in both adherent cell lines (MCA205 and McA-RH7777 in FIGS. 8-9, respectively) increased to a peak value of 40% higher than controls. There was also much more variability in the maximum change in caspase activation from control values noted in the adherent lines.

The analysis of caspase activation by NPS reveals some very interesting characteristics. When the caspase activation at various applied field strengths is plotted versus treatment energy, the curves are fairly uniform (FIG. 7). Nearly all of the pulse field strengths applied exhibited caspase activation peaks in the 10-20 J/mL range. This is very close to the 10-15 J/mL range of the cell viability ED50 showing a relationship between caspase activation energy and ablative efficacy. For treatment energies greater than 20 J/mL, the activated caspase fell to levels below control values, suggesting the majority of cells succumbed to necrosis at higher energy levels.

One unexpected result revealed in these studies is that the maximum increase in caspase activation was not observed for the highest field strengths tested of 25 and 30 kV/cm. Instead, the peak response was obtained in response to applied fields in the 12-20 kV/cm range and the response was smaller for both lower and higher field strengths (FIG. 7).

Annexin V/7-AAD Detection

Although caspase-3 activation can indicate the presence of caspase-3-dependent apoptosis, the inventors were interested in the overall percentage of cells that were succumbing to apoptosis verses some other cell death modality. In order to test this, an Annexin V/7-AAD flow cytometric based detection assay was used. PE Annexin V binds phosphatidylserine (PS), which is translocated from the inner to the outer leaflet of the plasma membrane during early apoptosis before loss of membrane integrity, making it a relatively good marker of early apoptosis. In contrast 7-AAD is only capable of crossing the cell membrane and binding to nucleic acids when cells are no longer viable and have become permeable. Detection of Annexin V alone indicates an early stage of apoptotic cell death where the cells are still viable and the membrane is still intact. The presence of both Annexin V and 7-AAD indicates a later stage of cell death where the cells are no-longer viable and the membranes have lost their integrity and become permeable.

After NPS treatment, the percent of cells undergoing these various stages of cell death at 3 hours (when we measure caspase-3 activation) and again at 24 hours (when we measure DAMPS) was measured to follow the progression. The percent of cells undergoing some stage of cellular demise shows an overall increase between 3 and 24 hours. Also, the percent of these cells that are in the latest stages of cell death clearly increased over time showing progression between 3 and 24 hours, particularly at energies above 15 J/mL.

There appears to be very little cell death in the MCA205 cell line at lower energies (5-15 J/mL). The percent of McA-RH7777 cells that are dead or dying at lower energies is also comparatively small to that seen at energies >25 J/mL. In both cell lines the percent of cells undergoing cell death increased significantly at 25 J/mL. In contrast, the Jurkat E6-1 cell line shows a steady increase across energies. Unlike the other two cell lines, there does not appear to be a particular energy threshold at which cells succumb.

Interestingly, the percentage of cells undergoing early apoptosis in the MCA205 and McA-RH7777 cell lines follows a different pattern across energies than do cells undergoing the later stages of cell death. The number of cells in the early stages of apoptosis, when cells are still viable, peaks at 15 J/mL and then drops off at 25 J/mL, showing the opposite pattern from those cells that are in the latest stages of cell death, which significantly increase in percentage at 25 J/mL. The Jurkat E6-1 cell line behavior again contrasts to the other two cell lines. The percent of cells in early apoptosis continues to increase as energy increases, similar in pattern to those at later stages of cell death.

Ecto-Calreticulin Expression Following NPS and Anthracycline Treatment

Flow cytometry was used to measure the percentage of tumor cells expressing cell surface calreticulin (ecto-CRT) after treatment with NPS at a range of energies or with two different anthracyclines (doxorubicin and mitoxantrone) at two different concentrations. Cell surface binding of anti-CRT was distinguished from internal CRT labeling by selecting cells that did not label with Zombie Aqua, indicating that they would be impermeable to the CRT antibody.

It was found that the percentage of cells expressing ecto-CRT increased as NPS treatment energy increased for all 3 cell lines reaching levels comparable to that resulting from anthracycline treatment between 25-50 J/mL. The percent of non-viable cells with membranes permeable to Zombie Aqua more than doubles after 15 J/mL. Additionally, there is a considerable drop-off in the total number of viable cells at 25 J/mL. Of the total cells that remained, more than 50% of the McA-RH7777 and Jurkat E6-1 cells expressed calreticulin on their surfaces at 25 J/mL. The percent of MCA205 cells that expressed calreticulin was lower but followed the same pattern of increase at 25 J/mL.

The degree of caspase 3/7 activation is highly cell dependent. For two adherent cell lines tested (MCA 205 mouse fibrosarcoma, McA-RH7777 rat hepatocarcinoma), a 20-30% increase in caspase 3 activation was observed following NPES treatment that peaks when 10 J/mL are applied at 25 kV/cm. At this field strength only 15 pulses are required to deliver this amount of energy. However, for the non-adherent Jurkat cell line, a much larger 420% increase is observed at a similar energy level.

The observation that all three cell lines exhibited a peak caspase 3 stimulation at a similar energy level suggests that it is the energy or charge delivered to the cells by the pulsed field that is a critical parameter in triggering apoptosis rather than pulse number or field strength.

ATP Secretion after NPS Treatment

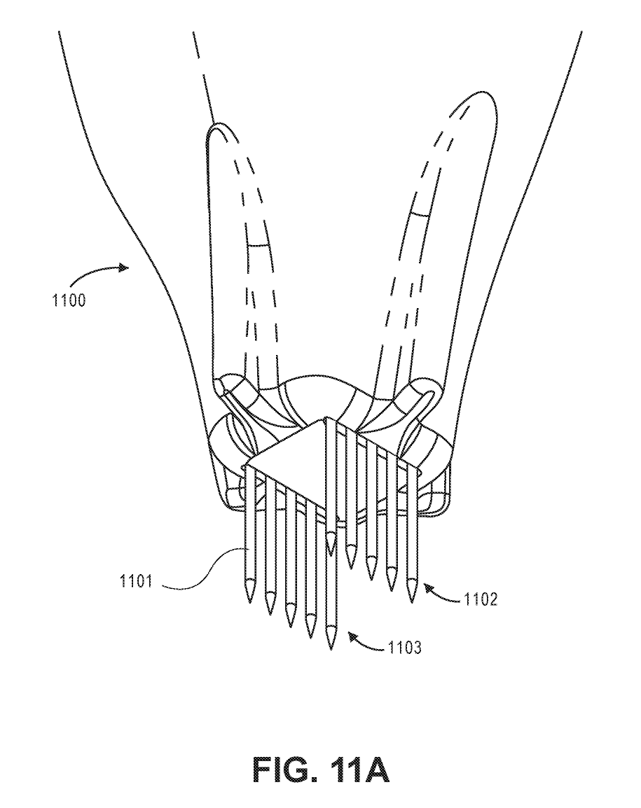

FIG. 10 includes charts showing ATP released from MCA205, McA-RH7777, and Jurkat E6-1 cells at different numbers of pulses.

The ATP released from both MCA205 and McA-RH7777 cells 24 hours after NPS treatment showed a well-defined peak at 15 J/mL (54-pulses; 15 kV/cm) with a sharp decline at 25 J/mL (see figure). The ATP release was highest at 15 J/mL in both cells lines and significantly so in the MCA205 compared with untreated cells. Cells treated with the higher concentration of doxorubicin (100 μM) released the second highest amount of ATP. The levels were also significantly higher in the MCA205 cell line than untreated cells. The mitoxantrone-treated cells released a comparatively small amount ATP at both high and low concentrations (4 and 10 μM).

Jurkat E6-1 ATP secretion levels were much lower than those observed from the adherent cell lines. ATP levels measured in the NPS or anthracycline treatment groups were not significantly different from background for any condition.

HMGB1 after NPS Treatment

The levels of HMGB1 24-hours post-NPS were energy-dependent and, similar to the expression of ecto-CRT, continued to increase as the treatment energy increased for all of the three cell lines. HMGB1 concentrations after NPS treatment reached or exceeded those measured after anthracycline treatment once energies reached between 10-25 J/mL.

The analysis of this caspase activation by NPES reveals why prior art treatments sometimes worked and sometimes did not.

An unexpected result revealed by these studies is that the maximum increase in caspase activation was not observed for the highest field strengths tested of 25 and 30 kV/cm. Instead the peak response was to applied fields in the 10-20 kV/cm range.

NPS is an effective, non-thermal therapy that can eliminate tumors without recurrence. The ER-stress response caused by the creation of nanopores and subsequent ATP release, as well as the ability to induce an adaptive immune response, lead us to suspect that the cell death modality of NPS was immunogenic cell death (ICD). When NPS is delivered at specific energies, it can induce some apoptotic cell death as well as the emission of three key markers of ICD.

Electrical Properties of NPS and Cell Viability

Cell viability is dependent on energy delivery and current flow across the cells. In experiments, the ED50 range of ablative efficacy is 10-15 J/mL for all field strengths above 10 kV/cm when the current flow across the cells is over 50 A/cm². When the current density is reduced to zero by increasing medium resistivity, cell viability increases dramatically to the point where no cells die after exposure to 16 J/mL. This increased viability in low conductivity media is somewhat controversial since some previous reports have observed that for low pulse numbers, the cell viability decreases in low conductivity media. Possible explanations of these contradictory observations include: 1) ionic dependence of membrane charging requirements to generate the nanopores; 2) Maxwell stress tensor inducing a stretching force on the membrane that increases in low conductivity, or, simply 3) the dependence of membrane properties on medium conductivity. The low conductivity solutions are quite likely not physiological and can have many effects on cells. One may conclude that NPS-induced cell death requires multiple pulses of current flow through cells.

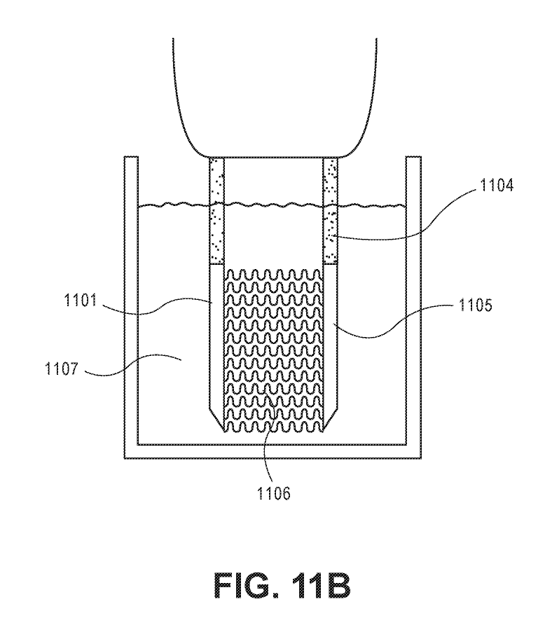

FIGS. 11A-11B are views of rows of needle electrodes that can provide electric fields similar to those of parallel plate electrodes shown in FIG. 1. In system 1100, electrodes 1101 include first row 1102 and second row 1103 of needle electrodes. The electrodes in each row are lined up with respect to one another. Rows 1102 and 1103 are separated by a distance between which cells in culture may be placed.

FIG. 11B shows needle electrodes 1101 plunged in liquid 1107 with cells. The upper end of the needle is surrounded with insulation 1104. Lower end 1105 of needle electrode 1105 is bare. Electric field 1106 passes between the rows of electrodes.

Needle electrodes in these dual row configurations, or three, four, or more row configurations, can approximate the results of parallel plate electrodes. Electrodes with a flattened blade instead of a sharp tip or other geometries/configurations of electrodes can also be used to good effect in order to approximate the electric field of parallel plate electrodes.

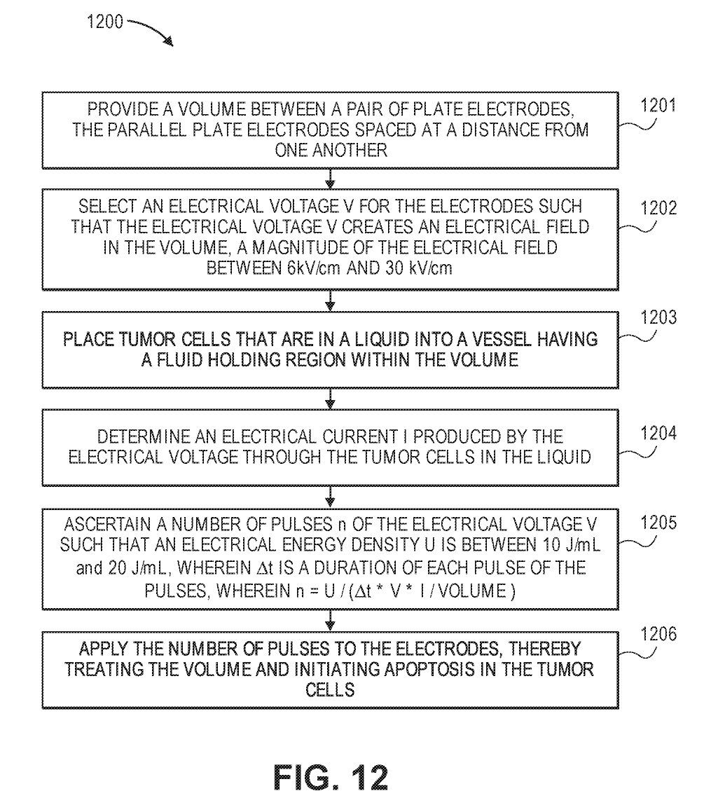

FIG. 12 is a flowchart illustrating process 1200 in accordance with an embodiment. In operation 1201, a volume between a pair of plate electrodes is provided, the parallel plate electrodes spaced at a distance from one another. In operation 1202, an electrical voltage V is selected for the electrodes such that the electrical voltage V creates an electrical field in the volume, a magnitude of the electrical field between 6 kV/cm and 30 kV/cm. In operation 1203, tumor cells that are in a liquid are placed into a vessel having a fluid holding region within the volume. In operation 1204, an electrical current I produced by the electrical voltage through the tumor cells in the liquid is determined. In operation 1205, a number of pulses n of the electrical voltage is ascertained such that an electrical energy density U is between 10 J/mL and 20 J/mL, wherein Δt is a duration of each pulse of the pulses, wherein n=U/(Δt*V*I/volume). In operation 1206, the number of pulses is applied to the electrodes, thereby treating the volume and initiating apoptosis in the tumor cells.

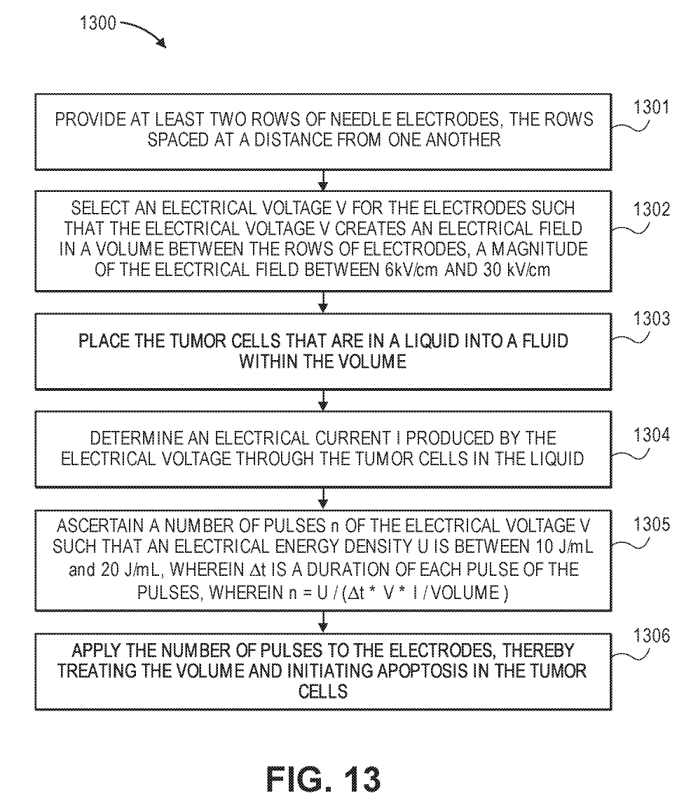

FIG. 13 is a flowchart illustrating process 1300 in accordance with an embodiment. In operation 1301, at least two rows of needle electrodes are provided, the rows spaced at a distance from one another. In operation 1302, an electrical voltage V is selected for the electrodes such that the electrical voltage V creates an electrical field in a volume between the rows of electrodes, a magnitude of the electrical field between 6 kV/cm and 30 kV/cm. In operation 1303, tumor cells that are in a liquid are placed within the volume. In operation 1304, an electrical current I produced by the electrical voltage through the tumor cells in the liquid is determined. In operation 1305, a number of pulses n of the electrical voltage is ascertained such that an electrical energy density U is between 10 J/mL and 20 J/mL, wherein Δt is a duration of each pulse of the pulses, wherein n=U/(Δt*V*I/volume). In operation 1306, the number of pulses is applied to the electrodes, thereby treating the volume and initiating apoptosis in the tumor cells.

Calculations

A culture of tumor cells on one day can have a different impedance than tumor cells in culture on another day. This can be because of the impedance of the tumor cells themselves as well as the number of ions in the culture preparation, etc.

Further, different cultures can be placed into different electroporation cuvettes or other vessels having fluid holding regions. Such vessels can have different parallel plate areas as well as different gaps (distances) between the plates. Standard electroporation cuvettes have gaps of 1.0 mm, 2.0 mm, and 4.0 mm. The surface area of one side of an electrode in a 4.0 mm cuvette can be 2.1 cm². For completeness, the volume defined by the plates is 0.84 mL.

To determine the resistive portion of impedance of tumor cells in culture, a small voltage Vtest (<500 V for a 1 cm gap) can be applied to the cuvette and a current Itest measured. The resistance of the culture is R=Vtest/Itest. Thus, for any voltage V applied to the cuvette, current will be I=V/R.

Given a treatment E-field, selected by a tumor type, one can calculate the number of pulses to be applied to the cuvette such that the total energy per unit volume is U=15 J/mL.

For example, if the resistance R is measured to be R=20Ω, then the number of pulses can be calculated for a desired electrical field of 10 kV/cm and other relevant parameters as:

$$\begin{matrix}V = 10 \frac{kV}{cm} * 0.4 cm \\ = 4 , 000 volts ( to be applied to cuvette electrodes )\end{matrix}$$

$$\begin{matrix}\# of pulses = U * volume / ( Δ t * {V}^{2} / R ) \\ = \frac{15 \frac{J}{mL} * 0.84 mL}{( 0.0000001 \frac{secs}{pulse} * {( 4 , 000 V )}^{2} / 20 Ω )} \\ = 158 pulses\end{matrix}$$

At 4 pps, such a treatment takes 39 seconds.

Other inputs can be determined as well. Pulse widths can be calculated given a predetermined number of pulses. For example, if it is determined that a treatment should only last 10 seconds at 4 pps for a total of 40 pulses, then:

$$\begin{matrix}Δ t = U * volume / ( n * {V}^{2} / R ) \\ = \frac{15 \frac{J}{mL} * 0.84 mL}{( 40 pulses * {( 4 , 000 V )}^{2} / 20 Ω )} \\ = 394 nsec\end{matrix}$$

One can also change the electric field, total energy, applied electrical field, frequency, etc. In an alternative case, the electric field is increased to 12 kV/cm. The calculation is then:

$$\begin{matrix}V = 12 \frac{kV}{cm} * 0.4 cm \\ = 4 , 800 volts ( to be applied to cuvette electrodes )\end{matrix}$$

$$\begin{matrix}Δ t = U * volume / ( n * V \hat{} 2 * R ) \\ = \frac{15 \frac{J}{mL} * 0.84 mL}{( 40 pulses * {( 4 , 800 V )}^{2} / 20 Ω )} \\ = 273 nsec\end{matrix}$$

A precision current limiter can be used in order to limit electrical current running through the sample. Non-equal pulse length durations may also be used.

Circulating Tumor Cells

Treating tumor cells in culture or liquid, as opposed to the tumor mass itself, can be lifesaving. Electroporation cuvettes and other vessels can be employed to impart the electric field pulses to the tumor cells.

Cancer that has metastasized through a subject's bloodstream may be treated using NPES's immune stimulation properties. For treatment, circulating tumor cells (CTCs) are isolated from the bloodstream and amassed in vial, test tube, or other suitable in vitro environment. In some cases, there may only be a few (e.g., 5, 10), tumor cells that are collected and amassed. Through this mass, an electric field is applied in order to treat the cells. This may or may not cause caspase expression or calreticulin to be expressed on the surface membranes of the tumor cells. The tumor cells may then be introduced back into the subject's bloodstream by injection, infusion, or otherwise.

In an alternative embodiment, single CTCs may also be isolated from the bloodstream, and each tumor cell treated individually. An automated system that captures CTCs in whole blood using iron nanoparticles coated with a polymer layer carrying biotin analogues and conjugated with antibodies for capturing CTCs can automatically capture the tumor cells, and a magnet and or centrifuge can separate them. After separation from the antibodies, the CTCs may be treated with NPES through a small capillary and then reintroduced to the patient's bloodstream.

The treated CTCs, with caspase or calreticulin expressed, can trigger an immune response in the subject against the cancer. A technical advantage of this method is that invasive surgery to remove a tumor may be avoided by simply treating CTCs. Further, a large number of tumors may be addressed at one time simply by triggering the body's own immune response. In vivo electroshocks, and their associated side effects, are avoided.

Immune Response

The immunogenic component of the response triggered by NPES has also been studied. In 2015, the inventors reported that nanoelectroablation of liver tumors in rats triggered a CD8-dependent immune response that blocked secondary tumor growth. It was reasoned that this immune response should also reduce metastasis in mice whose primary tumor was nanoelectroablated.

This hypothesis was tested by injecting live tumor cells into the tail vein of mice 4 weeks after the nsPEF-induced initiation of immunogenic apoptosis in one melanoma tumor (2000 pulses, 55 kV/cm, 100 ns). For a control we surgically removed tumors from mice on the same day that the tumors in other mice were treated with NPES.

Under conditions in which the lungs from mice whose primary tumor was surgically removed exhibited a mean of 17 metastases, lungs from mice whose tumor had been nanoelectroablated, had a mean of 4 metastases. This was significantly different with a p-value of p=0.03.

In addition, we measured the metastasis in a control group in which a single melanoma was ablated with NPES but no challenge with live tumor cells was carried out. We found that most of those animals had no metastasis 7 weeks after NPES treatment (avg=0.5 metastases/animal) and that was significantly different from the challenged group with p=0.4. These data certainly suggest that immunogenic apoptosis is reducing the degree of metastasis in these animals.

What is claimed is:

1. A method of treating abnormal cells, the method comprising:
providing a volume between a pair of parallel plate electrodes, the parallel plate electrodes spaced at a distance from one another;
selecting an electrical voltage V for the electrodes such that the electrical voltage V creates an electrical field in the volume, a magnitude of the electrical field at or above 6 kV/cm;
placing abnormal cells within the volume;
determining an electrical current I produced by the electrical voltage through the abnormal cells in the volume;
ascertaining a number of pulses n of the electrical voltage V such that an electrical energy density U is between 10 J/mL and 20 J/mL, wherein Δt is a duration of each pulse of the pulses, wherein n=U/(Δt*V*I/volume); and
applying the number of pulses to the electrodes, thereby treating the volume and initiating apoptosis in the abnormal cells.

2. The method of claim 1 further comprising:
changing the number of pulses n, during the applying, based on a feedback measurement of at least one prior pulse.

3. The method of claim 2 wherein the changing results in the number of pulses n becoming fewer.

4. The method of claim 2 wherein the changing results in the number of pulses n becoming larger.

5. The method of claim 1 further comprising:
changing the duration of each pulse Δt, during the applying, based on a feedback measurement of at least one prior pulse.

6. The method of claim 1 further comprising:
changing the electrical voltage V, during the applying, based on a feedback measurement of at least one prior pulse.

7. The method of claim 1 wherein the determining comprises:
energizing the electrodes with the electrical voltage; and
measuring the electrical current I.

8. The method of claim 1 wherein the determining comprises:
looking up the electrical current I from a memory.

9. The method of claim 1 wherein the parallel plate electrodes each have a surface area of ‘a’ and the distance is d, the volume being calculated by a*d.

10. The method of claim 1 wherein placing the abnormal cells within the volume includes placing liquid with the abnormal cells into a vessel having a fluid holding region within the volume.

11. The method of claim 10 wherein the vessel having a fluid holding region within the volume between the pair of electrodes includes an electroporation cuvette.

12. The method of claim 11 wherein the electroporation cuvette incorporates the parallel plate electrodes, each electrode having a surface area ‘a’ of 2.1 cm², the cuvette having a distance d of 0.4 cm between the electrodes for a volume of 0.84 mL.

13. The method of claim 1 wherein each pulse has a duration Δt of 100 ns, and the pulses are applied at 4 Hz to 10 Hz.

14. The method of claim 1 wherein the magnitude of the electrical field is between 6 kV/cm and 30 kV/cm.

15. The method of claim 1 wherein the abnormal cells are in a liquid culture.

16. The method of claim 1 further comprising:
drawing the abnormal cells from a subject during a biopsy; and
reintroducing the abnormal cells, after the initiation of apoptosis, into the subject.

17. A method of treating abnormal cells, the method comprising:
providing at least two rows of electrodes, the rows of electrodes spaced at a distance from one another;
selecting an electrical voltage V for the electrodes such that the electrical voltage V creates an electrical field in a volume between the rows of electrodes, a magnitude of the electrical field at or above 6 kV/cm;
placing abnormal cells within the volume;
determining an electrical current I produced by the electrical voltage through the abnormal cells in the volume;
ascertaining a number of pulses n of the electrical voltage V such that an electrical energy density U is between 10 J/mL and 20 J/mL, wherein Δt is a duration of each pulse of the pulses, wherein n=U/(Δt*V*I/volume); and
applying the number of pulses to the electrodes, thereby treating the volume and initiating apoptosis in the abnormal cells.

18. The method of claim 17 further comprising:
changing the number of pulses n, during the applying, based on a feedback measurement of at least one prior pulse.

19. The method of claim 18 wherein the changing results in the number of pulses n becoming fewer.

20. The method of claim 18 wherein the changing results in the number of pulses n becoming larger.

21. The method of claim 17 further comprising:
changing the duration of each pulse Δt, during the applying, based on a feedback measurement of at least one prior pulse.

22. The method of claim 17 further comprising:
changing the electrical voltage V, during the applying, based on a feedback measurement of at least one prior pulse.
